(19) United States
(12) Patent Application Publication
(10) Pub. No.: US 2017/0129869 A1
(43) Pub. Date: May 11, 2017

(54) AMORPHOUS FORM OF ELIGLUSTAT HEMITARTARATE

(71) Applicant: DR. REDDY'S LABORATORIES LIMITED, Hyderabad, TS (IN)

(72) Inventors: Dharma Jagannadha Rao VELAGA, Visakhapatnam (IN); Vishweshwar PEDDY, Hyderabad (IN); Sunitha VYALA, Hyderabad (IN)

(21) Appl. No.: 15/322,636

(22) PCT Filed: Jul. 3, 2015
(86) PCT No.: PCT/IB2015/055024

§ 371 Date: Dec. 28, 2016

(30) Foreign Application Priority Data
Jul. 3, 2014 (IN) 3293/CHE/2014

(51) Int. Cl. C07D 319/18; A61K 47/38; A61K 47/32; A61K 31/4025; A61K 9/10

(52) U.S. Cl. CPC C07D 319/18; A61K 31/4025; A61K 9/10; A61K 47/32; A61K 47/38

(57) ABSTRACT

The present application relates to the solid state forms of Eliglustat hemitartrate and the processes for the preparation thereof. The application further provides solid dispersion of Eliglustat hemitartrate having Eliglustat hemitartrate in amorphous form.

### INTRODUCTION

The present application relates to the solid state forms of Eliglustat hemitartrate and the processes for the preparation thereof.

Chemically Eliglustat is named N-[(1R,2R)-2-(2,3-dihydro-1,4-benzodioxin-6-yl)-2-hydroxy-1-(1-pyrrolidinylmethyl)ethyl]-Octanamide(2R,3R)-2,3-dihydroxybutanedioate and the hemitartarate salt of eliglustat has the structural formula as shown in Formula I.

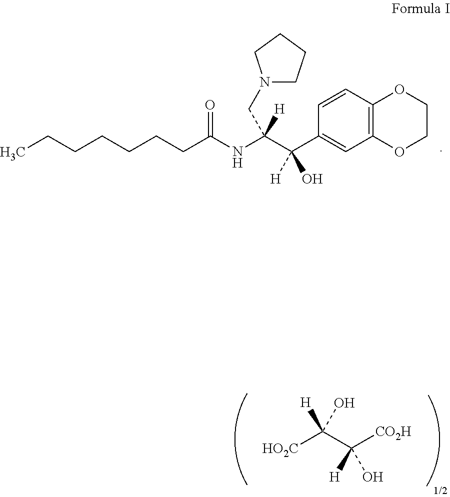

Eliglustat hemitartrate (Genz-112638), currently under development by Genzyme, is a glucocerebroside (glucosylceramide) synthase inhibitor for the treatment of Gaucher disease and other lysosomal storage disorders. Eliglustat hemitartrate is orally active with potent effects on the primary identified molecular target for type 1 Gaucher disease and other glycosphingolipidoses, appears likely to fulfill high expectations for clinical efficacy. Gaucher disease belongs to the class of lysosomal diseases known as glycosphingolipidoses, which result directly or indirectly from the accumulation of glycosphingolipids, many hundreds of which are derived from glucocerebroside. The first step in glycosphingolipid biosynthesis is the formation of glucocerebroside, the primary storage molecule in Gaucher disease, via glucocerebroside synthase (uridine diphosphate [UDP]—glucosylceramide glucosyl transferase). Eliglustat hemitartrate is based on improved inhibitors of glucocerebroside synthase, and is currently under development by Genzyme.

U.S. Pat. No. 7,196,205 discloses a process for the preparation of Eliglustat or a pharmaceutically acceptable salt thereof.

U.S. Pat. Nos. 6,855,830, 7,265,228, 7,615,573, 7,763,738, 8,138,353, U.S. patent application publication No. 2012/296088 discloses process for preparation of Eliglustat and intermediates thereof.

U.S. patent application publication No. 2013/137743 discloses (i) a hemitartrate salt of Eliglustat, (ii) a hemitartrate salt of Eliglustat, wherein at least 70% by weight of the salt is crystalline, (iii) a hemitartrate salt of Eliglustat, wherein at least 99% by weight of the salt is in a single crystalline form.

It has been disclosed earlier that the amorphous forms in a number of drugs exhibit different dissolution characteristics and in some cases different bioavailablity patterns compared to crystalline forms [Konne T., Chem pharm Bull., 38, 2003(1990)]. For some therapeutic indications one bioavailabihty pattern may be favoured over another. An amorphous form of Cefuroxime axetil is a good example for exhibiting higher bioavailability than the crystalline form.

Solid amorphous dispersions of drugs are known generally to improve the stability and solubility of drug products. However, such dispersions are generally unstable over time. Amorphous dispersions of drugs tend to convert to crystalline forms over time, which can lead to improper dosing due to differences of the solubility of crystalline drug material compared to amorphous drug material. The present invention, however, provides stable amorphous dispersions of eliglustat hemitartrate. Moreover, the present invention provides solid dispersions of eliglustat hemitartrate which may be reproduced easily and is amenable for processing into a dosage form.

There remains a need to provide solid state forms of eliglustat hemitartarate which are advantageous in a cost effective and environment friendly manner.

### SUMMARY

In the first embodiment, the present application provides an amorphous form of eliglustat hemitartarate.

In the second embodiment, the present application provides an amorphous form of eliglustat hemitartarate characterized by powder X-ray diffraction (PXRD) pattern substantially as illustrated by FIG. 1.

In the third embodiment, the present application provides a process for preparing amorphous form of eliglustat hemitartarate, which comprises;
a) providing a solution of eliglustat hemitartarate in a solvent;
b) removing solvent from a solution of eliglustat hemitartarate obtained in step a); and
c) recovering amorphous form of eliglustat hemitartarate.

In the fourth embodiment, the present application provides a solid dispersion comprising an amorphous form of eliglustat hemitartarate and one or more pharmaceutically acceptable carriers.

In the fifth embodiment, the present application provides a solid dispersion comprising an amorphous form of eliglustat hemitartarate and one or more pharmaceutically acceptable carriers characterized by powder X-ray diffraction (PXRD) substantially as illustrated by FIG. 5.

In the sixth embodiment, the present application provides a process for preparing a solid dispersion comprising an amorphous form of eliglustat hemitartarate and one or more pharmaceutically acceptable carriers, which comprises;
a) providing a solution of eliglustat hemitartrate and pharmaceutically acceptable carrier in a solution,
b) removing solvent from a solution obtained in step (a) and
c) recovering a solid dispersion comprising an amorphous form of eliglustat hemitartarate and one or more pharmaceutically acceptable carrier.

### BRIEF DESCRIPTION OF THE DRAWINGS

FIG. 1 is powder X-ray power diffraction (“PXRD”) pattern of amorphous form of eliglustat hemitartarate prepared according to Example 1.

FIG. 2 is powder X-ray power diffraction (“PXRD”) pattern of amorphous form of eliglustat hemitartarate prepared according to Example 2.

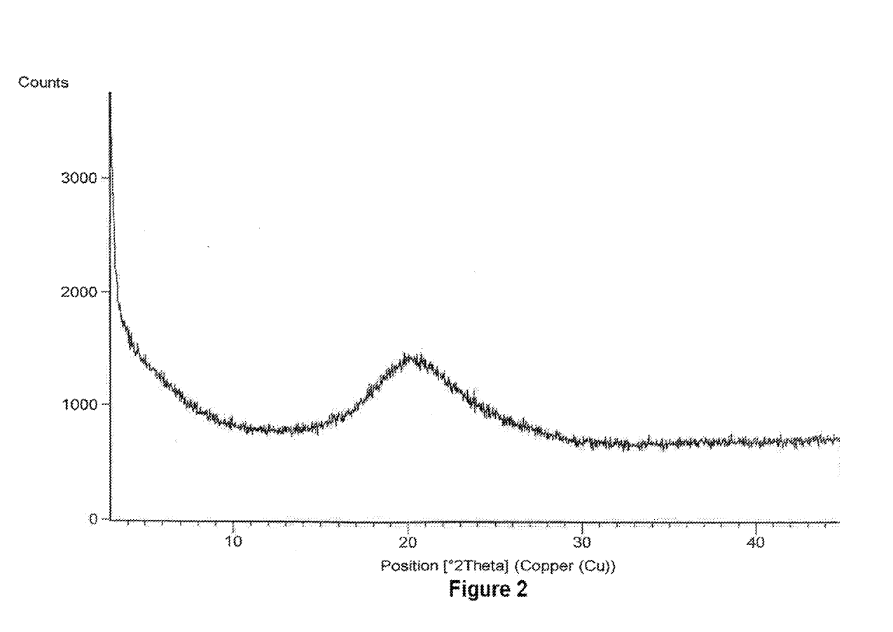

FIG. 3 is powder X-ray power diffraction (“PXRD”) pattern of a solid dispersion comprising an amorphous form of eliglustat hemitartarate and PVP-K30 prepared according to Example 4.

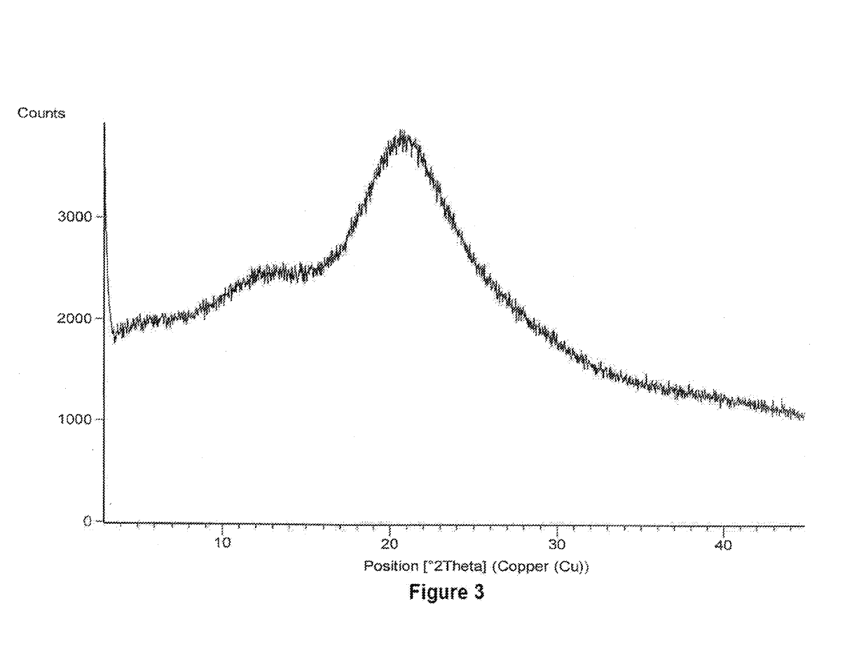

FIG. 4 is powder X-ray power diffraction (“PXRD”) pattern of a solid dispersion comprising an amorphous form of eliglustat hemitartarate and hydroxy propyl cellulose prepared according to Example 5.

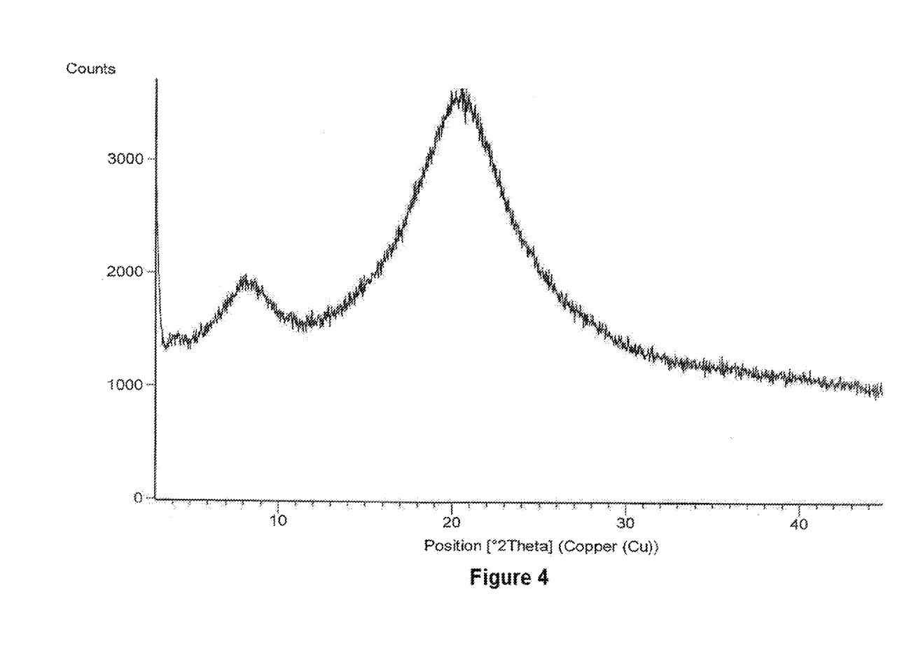

FIG. 5 is powder X-ray power diffraction (“PXRD”) pattern of a solid dispersion comprising an amorphous form of eliglustat hemitartarate and hydroxy propyl methyl cellulose prepared according to Example 6.

FIG. 6 is powder X-ray power diffraction (“PXRD”) pattern of a solid dispersion comprising an amorphous form of eliglustat hemitartarate and hydroxy propyl methyl cellulose prepared according to Example 12.

FIG. 7a is powder X-ray power diffraction (“PXRD”) pattern of a solid dispersion comprising an amorphous form of eliglustat hemitartarate, PVP K-30 and Syloid prepared according to Example 15.

FIG. 7b is powder X-ray power diffraction (“PXRD”) pattern of a solid dispersion comprising an amorphous form of eliglustat hemitartarate, PVP K-30 and Syloid prepared according to Example 15.

FIG. 8a is powder X-ray power diffraction (“PXRD”) pattern of a solid dispersion comprising an amorphous form of eliglustat hemitartarate, Copovidone and Syloid prepared according to Example 16.

FIG. 8b is powder X-ray power diffraction (“PXRD”) pattern of a solid dispersion comprising an amorphous form of eliglustat hemitartarate, Copovidone and Syloid prepared according to Example 16.

FIG. 9 is powder X-ray power diffraction (“PXRD”) pattern of a solid dispersion comprising an amorphous form of eliglustat hemitartarate and Syloid 244FP prepared according to Example 17.

### DETAILED DESCRIPTION

The present invention provides amorphous forms of eliglustat hemitartarate. Eliglustat or its hemitartarate salt which may be used as the input in the process for preparation of the solid states of the present application can be prepared by any process known in the art.

In the first embodiment, the present application provides an amorphous form of eliglustat hemitartarate.

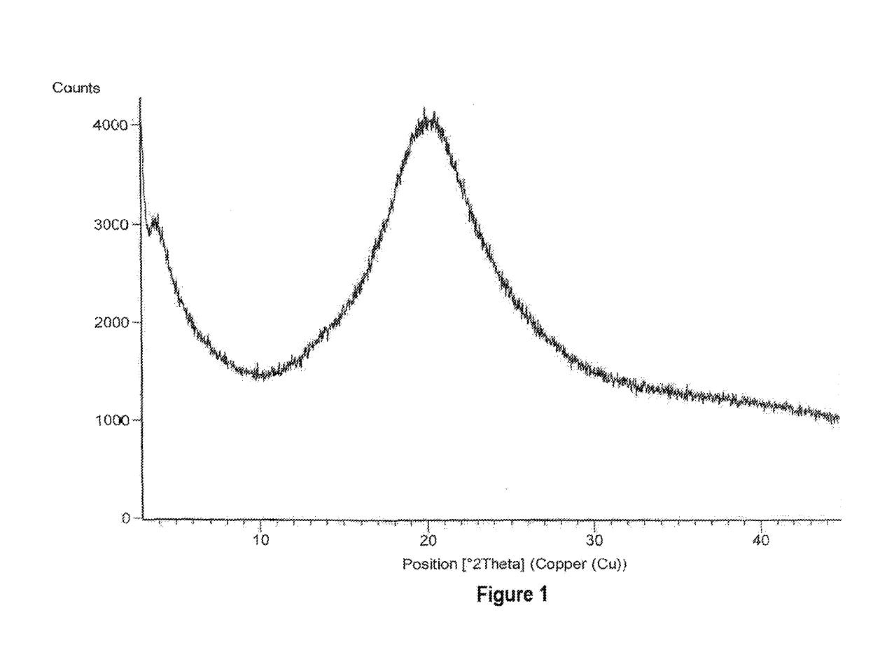

In the second embodiment, the present application provides an amorphous form of eliglustat hemitartarate characterized by powder X-ray diffraction (PXRD) pattern substantially as illustrated by FIG. 1.

In the third embodiment, the present application provides a process for preparing an amorphous form of eliglustat hemitartarate, which comprises;
a) providing a solution of eliglustat hemitartarate in a solvent;
b) removing solvent from a solution of eliglustat hemitartarate; and
c) recovering an amorphous form of eliglustat hemitartarate.
   Providing a solution in step a) includes:
i) direct use of a reaction mixture containing eliglustat hemitartarate that is obtained in the course of its synthesis; or
ii) direct use of reaction mixture containing eliglustat hemitartarate that is obtained by treating eliglustat with tartaric acid; or
ii) dissolving eliglustat hemitartarate in a solvent.

Any physical form of eliglustat hemitartarate may be utilized for providing the solution of eliglustat hemitartarate in step a).

Suitable solvents which can be used for dissolving the hemi tartrate salt of eliglustat include but are not limited to: alcoholic solvents such as methanol, ethanol, isopropyl alcohol, n-propanol, isoamyl alcohol and the like; halogenated hydrocarbons such as dichloromethane, 1,2-dichloroethane, chloroform, carbon tetrachloride and the like; ketones such as acetone, ethyl methyl ketone, methyl isobutyl ketone and the like; esters such as ethyl acetate, n-propyl acetate, n-butyl acetate, t-butyl acetate and the like; ethers such as diethyl ether, dimethyl ether, di-isopropyl ether, 1,4-dioxane and the like; hydrocarbons such as toluene, xylene and the like; nitriles such as acetonitrile, propionitrile and the like; and any mixtures of two or more thereof.

After dissolution in step (a), the obtained solution may be optionally filtered to remove any insoluble particles. Suitable techniques to remove insoluble particles are filtration, centrifugation, decantation, and any other known techniques in the art. The solution can be filtered by passing through paper, glass fiber, or other membrane material, or a clarifying agent such as celite. Depending upon the equipment used and the concentration and temperature of the solution, the filtration apparatus may need to be preheated to avoid premature precipitation of solid. Step (b) involves removing solvent from a solution of eliglustat hemitartarate.
Suitable techniques which can be used for the removal of solvent include but not limited to evaporation, flash evaporation, simple evaporation, rotational drying, spray drying, agitated thin-film drying, agitated nutsche filter drying, pressure nutsche filter drying, freeze-drying or any other suitable technique known in the art.
The solvent may be removed, optionally under reduced pressures, at temperatures less than about 100° C., less than about 75° C., less than about 60° C., less than about 50° C., or any other suitable temperatures. Step (c) involves recovering an amorphous form of eliglustat hemitartarate. The said recovery can be done by using the processes known in the art.
The solid obtained from step c) may be collected by using techniques such as by scraping, or by shaking the container, or other techniques specific to the equipment used. The isolated solid may be optionally further dried to afford an amorphous form of eliglustat hemitartrate.
The resulting compound in step (c) may be optionally further dried. Drying can be carried out in a tray dryer, vacuum oven, air oven, cone vacuum dryer, rotary vacuum dryer, fluidized bed dryer, spin flash dryer, flash dryer, or the like. The drying can be carried out at temperatures of less than about 60° C., less than about 50° C., less than about 40° C., less than about 30° C., less than about 20° C., or any other suitable temperatures; at atmospheric pressure or under a reduced pressure; as long as the eliglustat hemitartrate is not degraded in its quality. The drying can be carried out for any desired times until the required product quality is achieved. Suitable time for drying can vary from few minutes to several hours for example from about 30 minutes to about 24 or more hours.

In the fourth embodiment, the present application provides a solid dispersion comprising an amorphous form of eliglustat hemitartarate and one or more pharmaceutically acceptable carriers.

Solid dispersion as used herein refers to the dispersion of one or more active ingredients in an inert excipient or matrix (carrier), where the active ingredients could exist in finely crystalline, solubilized or amorphous state (Sareen et al., 2012 and Kapoor et al., 2012). Solid dispersion consists of two or more than two components, generally a carrier polymer and drug optionally along with stabilizing agent (and/or surfactant or other additives). The most important role of the added polymer in solid dispersion is to reduce the molecular mobility of the drug to avoid the phase separation and re-crystallization of drug during storage. The increase in solubility of the drug in solid dispersion is mainly because drug remains in amorphous form which is associated with a higher energy state as compared to crystalline counterpart and due to that it required very less external energy to dissolve.

A solid dispersion is a molecular dispersion of a compound, particularly a drug substance within a carrier matrix. Formation of a molecular dispersion provides a means of reducing the particle size to nearly molecular levels (i.e. there are no particles). As the carrier dissolves, the drug is exposed to the dissolution media as fine particles that are amorphous, which can dissolve and be absorbed more rapidly than larger particles.

In general, the term “solid dispersion” refers to a system in a solid state comprising at least two components, wherein one component is dispersed throughout the other component or components. The term “amorphous solid dispersion” as used herein, refers to stable solid dispersions comprising amorphous drug substance and a carrier matrix. By “amorphous drug substance,” it is meant that the amorphous solid dispersion contains drug substance in a substantially amorphous solid state form i.e. at least 80% of the drug substance in the dispersion is in an amorphous form. More preferably at least 90% and most preferably at least 95% of the drug substance in the dispersion is in amorphous form.

The solid dispersions of eliglustat hemitartrate of the present invention can be made by any of numerous methods that result in an amorphous solid dispersion of eliglustat hemitartrate. Several approaches can be used for the preparation of solid dispersion which includes spray drying, fusion method, solvent evaporation, hot-melt extrusion, particle size reduction, supercritical fluid (SCF) processes, kneading, inclusion complexes, electrostatic spinning method and surface-active carriers.

The dispersing agent is typically composed of a pharmaceutically acceptable substance that does not substantially interfere with the pharmaceutical action of eliglustat hemitartrate. The phrase “pharmaceutically acceptable” is employed herein to refer to those substances which are, within the scope of sound medical judgment, suitable for use in contact with the tissues of human beings and animals without excessive toxicity, irritation, allergic response, or other problem or complication, commensurate with a reasonable benefit/risk ratio. In some embodiments, the carrier is a solid at room temperature (e.g., about 25 oC). In further embodiments, the carrier melts at a temperature between about 30 and 100 oC. In further embodiments, the carrier is soluble in an organic solvent.

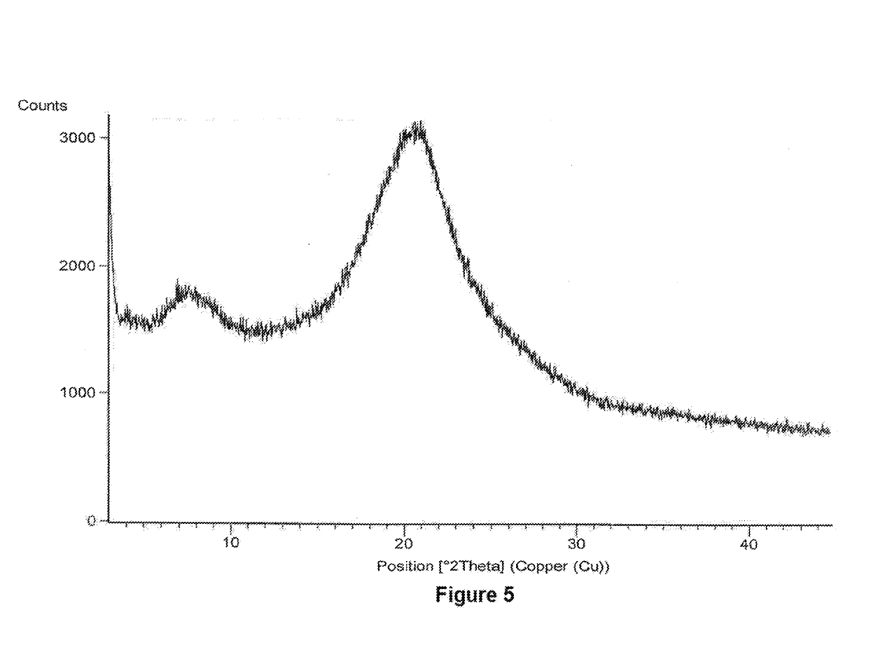

In the fifth embodiment, the present application provides a solid dispersion comprising an amorphous form of eliglustat hemitartarate and one or more pharmaceutically acceptable carriers characterized by powder X-ray diffraction (PXRD) substantially as illustrated by FIG. 5.

In the sixth embodiment, the present application provides a process for preparing a solid dispersion comprising an amorphous form of eliglustat hemitartarate and one or more pharmaceutically acceptable carriers, which comprises;
a) providing a solution of eliglustat hemitartrate and pharmaceutically acceptable carrier in a solution,
b) removing solvent from a solution obtained in step (a); and
c) recovering a solid dispersion comprising an amorphous form of eliglustat hemitartarate and one or more pharmaceutically acceptable carrier.
   Providing a solution in step a) includes:
i) direct use of a reaction mixture containing eliglustat hemitartarate that is obtained in the course of its synthesis; or
ii) direct use of a reaction mixture containing eliglustat hemitartarate that is obtained by treating eliglustat with tartaric acid; or
ii) dissolving eliglustat hemitartarate and pharmaceutically acceptable carrier in a solvent.

Any physical form of eliglustat hemitartarate may be utilized for providing the solution of eliglustat hemitartarate in step (a).

Suitable pharmaceutically acceptable carriers that are dispersing agents which can be used in step (a) include, but are not limited to: diluents such as starches, pregelatinized starches, lactose, powdered celluloses, microcrystalline celluloses, dicalcium phosphate, tricalcium phosphate, mannitol, sorbitol, sugar and the like; binders such as acacia, guar gum, tragacanth, gelatin, polyvinylpyrrolidones, hydroxypropyl celluloses, hydroxypropyl methylcelluloses, pregelatinized starches and the like; disintegrants such as starches, sodium starch glycolate, pregelatinized starches, crospovidones, croscarmellose sodium, colloidal silicon dioxide and the like; lubricants such as stearic acid, magnesium stearate, zinc stearate and the like; glidants such as colloidal silicon dioxide (Syloid, Aerosil, Cab-o-sil etc.) and the like; solubility or wetting enhancers such as anionic or cationic or neutral surfactants; complex forming agents such as various grades of cyclodextrins and resins; release rate controlling agents such as hydroxypropyl celluloses, hydroxymethyl celluloses, hydroxypropyl methylcelluloses, ethylcelluloses, methylcelluloses, various grades of methyl methacrylates, waxes and the like. Other pharmaceutically acceptable excipients that are of use include but are not limited to film formers, plasticizers, colorants, flavoring agents, sweeteners, viscosity enhancers, preservatives, antioxidants, and the like.

Suitable solvents which can be used for dissolving the hemi tartrate salt of eliglustat include but are not limited to: alcoholic solvents such as methanol, ethanol, isopropyl alcohol, n-propanol, isoamyl alcohol and the like; halogenated hydrocarbons such as dichloromethane, 1,2-dichloroethane, chloroform, carbon tetrachloride and the like; ketones such as acetone, ethyl methyl ketone, methyl isobutyl ketone and the like; esters such as ethyl acetate, n-propyl acetate, n-butyl acetate, t-butyl acetate and the like; ethers such as diethyl ether, dimethyl ether, di-isopropyl ether, 1,4-dioxane and the like; hydrocarbons such as toluene, xylene and the like; nitriles such as acetonitrile, propionitrile and the like; water and any mixtures of two or more thereof.

After dissolution in step (a), optionally undissolved particles, if any, may be removed suitably by filtration, centrifugation, decantation, and any other known techniques. The solution can be filtered by passing through paper, glass fiber, or other membrane material, or a clarifying agent such as celite. Depending upon the equipment used and the concentration and temperature of the solution, the filtration apparatus may need to be preheated to avoid premature crystallization.

Step (b) involves removing solvent from a solution obtained in step (a);

Suitable techniques which can be used for the removal of solvent include but not limited to evaporation, flash evaporation, simple evaporation, rotational drying, spray drying, agitated thin-film drying, agitated nutsche filter drying, pressure nutsche filter drying, freeze-drying or any other technique known in the art.

Step (c) involves recovering a solid dispersion comprising an amorphous form of eliglustat hemitartarate and one or more pharmaceutically acceptable carriers. The said recovery can be by using the processes known in the art.

The resulting compound obtained in step (c) may be optionally further dried. Drying can be carried out in a tray dryer, vacuum oven, air oven, cone vacuum dryer, rotary vacuum dryer, fluidized bed dryer, spin flash dryer, flash dryer, or the like. The drying can be carried out at temperatures of less than about 60° C., less than about 50° C., less than about 40° C., less than about 30° C., less than about 20° C., or any other suitable temperatures; at atmospheric pressure or under a reduced pressure; as long as the eliglustat hemitartrate is not degraded in its quality. The drying can be carried out for any desired times until the required product quality is achieved. Suitable time for drying can vary from few minutes to several hours for example from about 30 minutes to about 24 or more hours.

The amount of eliglustat hemitartrate in the solid dispersions of the present invention ranges from about 0.1% to about 90%, by weight, of the solid dispersion; or from about 10% to about 70%, by weight, of the solid dispersion; or from about 20% to about 60%, by weight, of the solid dispersion; or from about 20% to about 40%, by weight, of the solid dispersion; or about 30%, by weight, of the solid dispersion. In some aspects, the weight ratio of eliglustat hemitartrate to carrier is about 1:99 to about 99:1. In some aspects, the weight ratio of eliglustat hemitartrate to carrier is about 1:99 to about 75:25 or about 1:99 to about 60: 40. In further aspects, the weight ratio of eliglustat hemitartrate to carrier is about 1:99 to about 15:85; about 1:99 to about 10:90; or about 1:99 to about 5:95. In further aspects, the weight ratio of eliglustat hemitartrate to carrier is about 25:75 to about 75:25, about 40:60 to about 60:40 or about 1:1 or about 2:1. Typically eliglustat hemitartrate and carrier medium are present in a ratio by weight with the solvent of 1:0.1 to 1:20.

The dried product be it amorphous eliglustat hemitartrate or solid dispersion comprising amorphous eliglustat hemitartrate may be optionally subjected to a particle size reduction procedure to produce desired particle sizes and distributions. Milling or micronization may be performed before drying, or after the completion of drying of the product. Equipment that may be used for particle size reduction include, without limitation thereto, ball mills, roller mills, hammer mills, and jet mills.

In another general aspect, there is provided amorphous form of eliglustat hemitartrate or solid dispersion comprising amorphous form of eliglustat hemitartrate having particle size distributions wherein D₉₀ is less than about 500 microns, preferably less than about 200 microns, more preferably less than about 100 microns, most preferably less than 10 microns.

In an aspect, the present application provides pharmaceutical formulations comprising an amorphous form of eliglustat hemitartrate or solid dispersion comprising amorphous form of eliglustat hemitartrate, together with one or more pharmaceutically acceptable excipients. Eliglustat hemitartrate together with one or more pharmaceutically acceptable excipients of the present application may be formulated as: solid oral dosage forms such as, but not limited to, powders, granules, pellets, tablets, and capsules; liquid oral dosage forms such as, but not limited to, syrups, suspensions, dispersions, and emulsions; and injectable preparations such as, but not limited to, solutions, dispersions, and freeze dried compositions. Formulations may be in the forms of immediate release, delayed release, or modified release. Further, immediate release compositions may be conventional, dispersible, chewable, mouth dissolving, or flash melt preparations, and modified release compositions that may comprise hydrophilic or hydrophobic, or combinations of hydrophilic and hydrophobic, release rate controlling substances to form matrix or reservoir or combination of matrix and reservoir systems. The compositions may be prepared using any one or more of techniques such as direct blending, dry granulation, wet granulation, and extrusion and spheronization. Compositions may be presented as uncoated, film coated, sugar coated, powder coated, enteric coated, and modified release coated.

Pharmaceutically acceptable excipients that are useful in the present application include, but are not limited to: diluents such as starches, pregelatinized starches, lactose, powdered celluloses, microcrystalline celluloses, dicalcium phosphate, tricalcium phosphate, mannitol, sorbitol, sugar, and the like; binders such as acacia, guar gum, tragacanth, gelatin, polyvinylpyrrolidones, hydroxypropyl celluloses, hydroxypropyl methyl celluloses, pregelatinized starches, and the like; disintegrants such as starches, sodium starch glycolate, pregelatinized starches, crospovidones, croscarmellose sodium, colloidal silicon dioxide, and the like; lubricants such as stearic acid, magnesium stearate, zinc stearate, and the like; glidants such as colloidal silicon dioxide and the like; solubility or wetting enhancers such as anionic, cationic, or neutral surfactants; complex forming agents such as various grades of cyclodextrins and resins; and release rate controlling agents such as hydroxypropyl celluloses, hydroxymethyl celluloses, hydroxypropyl methylcelluloses, ethylcelluloses, methylcelluloses, various grades of methyl methacrylates, waxes, and the like. Other pharmaceutically acceptable excipients that are useful include, but are not limited to, film formers, plasticizers, colorants, flavoring agents, sweeteners, viscosity enhancers, preservatives, antioxidants, and the like.

The pharmaceutical dosage form according to the present invention may be is coated with one or more coating materials or uncoated. The coating materials are not particularly limited and are known to the person skilled in the art.

Solid states of eliglustat hemitartarate of the present application are characterized by its PXRD pattern. All PXRD data reported herein were obtained using Cu Kα radiation, having the wavelength 1.541 A, and were obtained using a PanAlytical, Powder X-ray Diffractometer.

The D10, D50, and D90 values are useful ways for indicating a particle size distribution. D90 refers to at least 90 volume percent of the particles having a size smaller than the said value. Likewise, D10 refers to 10 volume percent of the particles having a size smaller than the said value. D50 refers to 50 volume percent of the particles having a size smaller than the said value. Methods for determining D10, D50, and D90 include laser diffraction, such as using equipment from Malvern Instruments Ltd. of Malvern, Worcestershire, United Kingdom.

Although the exemplified procedures herein illustrate the practice of the present invention in some of its embodiments, the procedures should not be construed as limiting the scope of the invention. Modifications from consideration of the specification and examples within the ambit of current scientific knowledge will be apparent to one skilled in the art.

### Definitions

The following definitions are used in connection with the present application unless the context indicates otherwise.

“Amorphous form” as used herein refers to a solid state wherein the amorphous content with in the said solid state is at least about 35% or at least about 40% or at least about 45% or at least about 50% or at least about 55% or at least about 60% or at least about 65% or at least about 70% or at least about 75% or at least about 80% or at least about 85% or at least about 90% or at least about 95% or at least about 96% or at least about 97% or at least about 98% or at least about 99% or about 100%.

An “alcohol” is an organic compound containing a carbon bound to a hydroxyl group. “C1-C6 alcohols” include, but are not limited to, methanol, ethanol, 2-nitroethanol, 2-fluoroethanol, 2,2,2-trifluoroethanol, hexafluoroisopropyl alcohol, ethylene glycol, 1-propanol, 2-propanol (isopropyl alcohol), 2-methoxyethanol, 1-butanol, 2-butanol, i-butyl alcohol, t-butyl alcohol, 2-ethoxyethanol, diethylene glycol, 1-, 2-, or 3-pentanol, neo-pentyl alcohol, t-pentyl alcohol, isoamyl alcohol, diethylene glycol monomethyl ether, diethylene glycol monoethyl ether, cyclohexanol, phenol, glycerol, or the like.

An “aliphatic hydrocarbon” is a liquid hydrocarbon compound, which may be linear, branched, or cyclic and may be saturated or have as many as two double bonds. A liquid hydrocarbon compound that contains a six-carbon group having three double bonds in a ring is called “aromatic.” Examples of “C5-C8 aliphatic or aromatic hydrocarbons” include, but are not limited to, isopentane, neopentane, isohexane, 3-methylpentane, 2,3-dimethylbutane, neohexane, isoheptane, 3-methylhexane, neoheptane, 2,3-dimethylpentane, 2,4-dimethylpentane, 3,3-dimethylpentane, 3-ethylpentane, 2,2,3-trimethylbutane, n-octane, isooctane, 3-methylheptane, neooctane, methylcyclohexane, cycloheptane, petroleum ethers, benzene toluene, ethylbenzene, m-xylene, o-xylene, p-xylene, trimethylbenzene, chlorobenzene, fluorobenzene, trifluorotoluene, anisole, or any mixtures thereof.

An “ester” is an organic compound containing a carboxyl group —(C═O)—O— bonded to two other carbon atoms. “C3-C6 esters” include, but are not limited to, ethyl acetate, n-propyl acetate, n-butyl acetate, isobutyl acetate, t-butyl acetate, ethyl formate, methyl acetate, methyl propanoate, ethyl propanoate, methyl butanoate, ethyl butanoate, or the like.

An “ether” is an organic compound containing an oxygen atom —O— bonded to two other carbon atoms. “C2-C6 ethers” include, but are not limited to, diethyl ether, diisopropyl ether, methyl t-butyl ether, glyme, diglyme, tetrahydrofuran, 2-methyltetrahydrofuran, 1,4-dioxane, dibutyl ether, dimethylfuran, 2-methoxyethanol, 2-ethoxyethanol, anisole, or the like.

A “halogenated hydrocarbon” is an organic compound containing a carbon bound to a halogen. Halogenated hydrocarbons include, but are not limited to, dichloromethane, 1,2-dichloroethane, trichloroethylene, perchloroethylene, 1,1,1-trichloroethane, 1,1,2-trichloroethane, chloroform, carbon tetrachloride, or the like.

A “ketone” is an organic compound containing a carbonyl group —(C═O)— bonded to two other carbon atoms. “C3-C6 ketones” include, but are not limited to, acetone, ethyl methyl ketone, diethyl ketone, methyl isobutyl ketone, ketones, or the like.

A “nitrile” is an organic compound containing a cyano —(C≡N) bonded to another carbon atom. “C2-C6 Nitriles” include, but are not limited to, acetonitrile, propionitrile, butanenitrile, or the like.

All percentages and ratios used herein are by weight of the total composition and all measurements made are at about 25° C. and about atmospheric pressure, unless otherwise designated. All temperatures are in degrees Celsius unless specified otherwise. As used herein, “comprising” means the elements recited, or their equivalents in structure or function, plus any other element or elements which are not recited. The terms “having” and “including” are also to be construed as open ended. All ranges recited herein include the endpoints, including those that recite a range “between” two values. Whether so indicated or not, all values recited herein are approximate as defined by the circumstances, including the degree of expected experimental error, technique error, and instrument error for a given technique used to measure a value.

Certain specific aspects and embodiments of the present application will be explained in greater detail with reference to the following examples, which are provided only for purposes of illustration and should not be construed as limiting the scope of the application in any manner. Reasonable variations of the described procedures are intended to be within the scope of the present invention. While particular aspects of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

### EXAMPLES

### Example 1

### Preparation of Amorphous Form of Eliglustat Hemitartarate

500 mg of eliglustat hemitartarate was dissolved in 14 mL of dichloromethane at 26° C. and stirred for 15 min. The solution is filtered to remove the undissolved particles and the filtrate is distilled under reduced pressure at 45° C. After distillation the solid was dried under vacuum at 45° C.

### Example 2

### Preparation of Amorphous Form of Eliglustat Hemitartarate

500 mg of eliglustat hemitartarate was dissolved in 70 mL of ethanol and stirred for 15 min at 25°-30° C. The solution is filtered to remove the undissolved particles and the filtrate is distilled under reduced pressure at 48° C. After distillation the solid was dried under vacuum at 48° C.

### Example 3

### Preparation of Amorphous Form of Eliglustat Hemitartarate

500 mg of eliglustat hemitartarate was dissolved in 20 mL of methanol and stirred for 15 min at 25°-30° C. The solution is filtered to remove the undissolved particles and the filtrate is distilled under reduced pressure at 48° C. After distillation the solid was dried under vacuum at 48° C.

### Example 4

### Preparation of a Solid Dispersion Comprising an Amorphous Form of Eliglustat Hemitartarate and PVP-K30

500 mg of eliglustat hemitartarate and 500 mg of PVP-K30 was dissolved in 20 mL of methanol and stirred for 10 min at 25°-30° C. The solution is filtered to remove the undissolved particles and the filtrate is distilled under reduced pressure at 48° C. After distillation the solid is dried under vacuum at 48° C.

### Example 5

### Preparation of a Solid Dispersion Comprising an Amorphous Form of Eliglustat Hemitartarate and Hydroxy Propyl Cellulose

500 mg of eliglustat hemitartarate and 500 mg of hydroxy propyl cellulose was dissolved in 30 ml of methanol and stirred for 10 min at 25°-30° C. The solution is distilled under reduced pressure at 49° C. After distillation the solid is dried under vacuum at 49° C.

### Example 6

### Preparation of a Solid Dispersion Comprising an Amorphous Form of Eliglustat Hemitartarate and Hydroxy Propyl Methyl Cellulose

500 mg of eliglustat hemitartarate and 500 mg of hydroxy propyl methyl cellulose was dissolved in 30 mL of methanol and stirred for 10 min at 25°-30° C. The solution is distilled under reduced pressure at 48° C. After distillation the solid is dried under vacuum at 48° C.

### Example 7

### Preparation of Amorphous Form of Eliglustat Hemitartarate

3 g of eliglustat hemitartarate was dissolved in 75 mL of methanol and stirred at 25° C. for dissolution. The solution was filtered to remove the undissolved particles and the filtrate is subjected for spray drying at inlet temperature of 70° C. and outlet temperature of 42° C. to afford the title compound.

### Example 8

### Preparation of Amorphous Form of Eliglustat Hemitartarate

500 mg of eliglustat hemitartarate was dissolved in 30 mL of isopropanol and stirred at 56° C. for dissolution. The solution was filtered to remove the undissolved particles and the filtrate is subjected to complete distillation under reduced pressure and drying at about 56° C. to afford the title compound.

### Example 9

### Preparation of Amorphous Form of Eliglustat Hemitartarate

1 g of eliglustat hemitartarate was provided in 40 mL of ethyl acetate and stirred at about 63° C. Then methanol (5 mL) is added at the same temperature to obtain clear solution which was filtered to remove the undissolved particles. Then additional quantity of methanol (5 mL) is added to the filtrate and the filtrate was again filtered to remove particles. The obtained filtrate was subjected to complete distillation under reduced pressure and drying at about 57° C. to afford the title compound.

### Example 10

### Preparation of Amorphous Form of Eliglustat Hemitartarate

1 g of eliglustat hemitartarate was provided in 40 mL of acetone and stirred at about 55° C. followed by addition of methanol (15 mL). The mixture is stirred at 55° C. for clear solution and filtered to remove the undissolved particles. The obtained filtrate was subjected to complete distillation under reduced pressure and drying at about 57° C. to afford the title compound.

### Example 11

### Preparation of Amorphous Form of Eliglustat Hemitartarate

1 g of eliglustat hemitartarate was provided in 25 mL of isopropyl alcohol and 25 mL of ethanol. The mixture was stirred at about 58° C. for dissolution and filtered to remove the undissolved particles. The obtained filtrate was subjected to complete distillation under reduced pressure and drying at about 57° C. to afford the title compound.

### Example 12

### Preparation of Amorphous Form of Eliglustat Hemitartarate

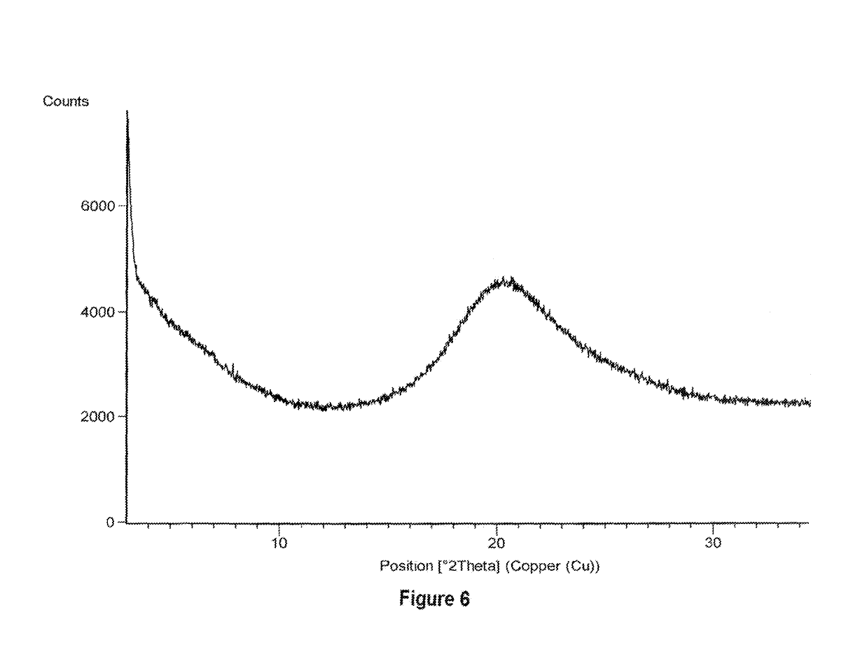

5 g of eliglustat hemitartarate was provided in 300 mL of isopropyl alcohol and stirred at about 59° C. for dissolution. The solution was filtered to remove the undissolved particles and the filtrate is subjected for spray drying at inlet temperature of 65° C. and outlet temperature of 37° C. to afford the title compound according to FIG. 6

### Example 13

### Preparation of a Solid Dispersion Comprising an Amorphous Form of Eliglustat Hemitartarate and Copovidone

500 mg of eliglustat hemitartarate and 500 mg of Copovidone were dissolved in 30 mL of methanol and stirred for clear solution, then filtered to make it particle free. The solvent from the filtrate was evaporated under reduced pressure at 45° C. and obtained solid was subjected to drying at 45° C. to afford the title solid. The resulting dispersion was found to be amorphous by X-ray powder diffraction.

### Example 14

### Preparation of a Solid Dispersion Comprising an Amorphous Form of Eliglustat Hemitartarate and Copovidone

2 g of eliglustat hemitartarate and 2 g of Copovidone were dissolved in 100 mL of methanol and stirred for clear solution, then filtered to make it particle free. The solvent from the filtrate was subjected to spray drying at inlet temperature of 70 at 45° C. and outlet temperature of 42° C. to afford the title compound. The resulting dispersion was found to be amorphous by X-ray powder diffraction.

### Example 15

### Preparation of a Solid Dispersion Comprising an Amorphous Form of Eliglustat Hemitartarate

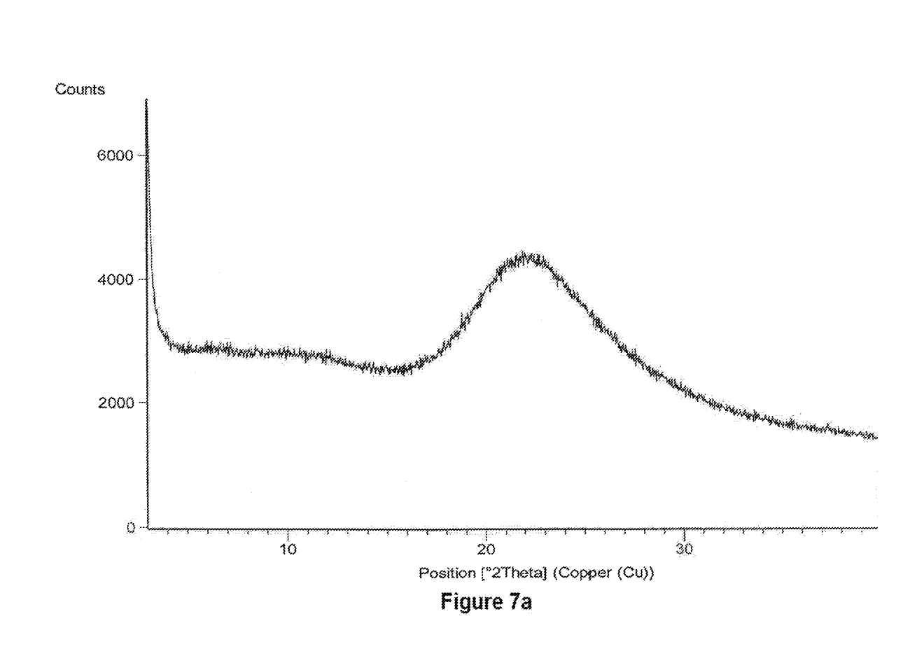

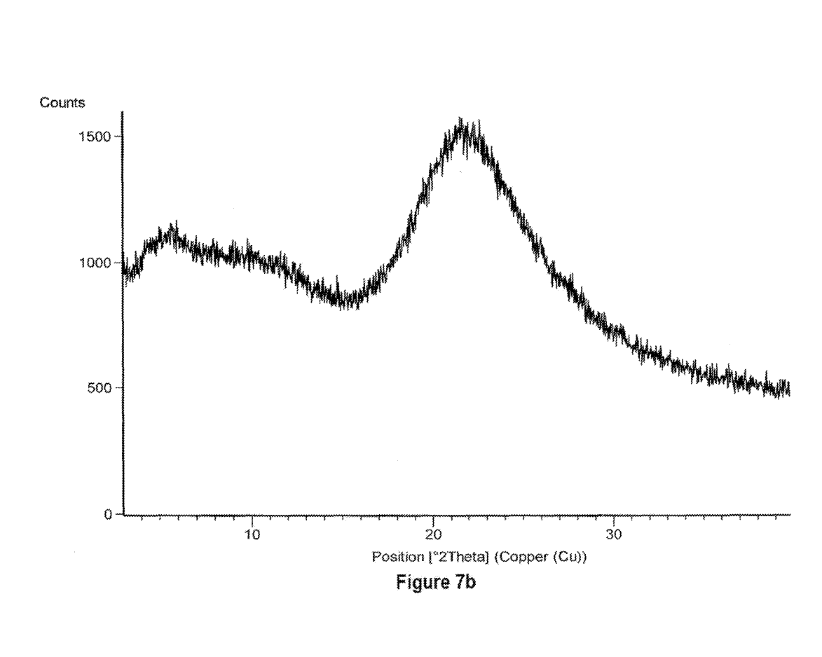

2 g of eliglustat hemitartarate was charged in 40 mL of methanol followed by addition of 2 g of PVP K-30. The mixture was stirred for clear solution and filtered to make it particle free, the bed was washed with 20 mL of methanol. Then 2 g of Syloid is added to the filtrate and filtrate is subjected to distillation under reduced pressure at about 57° C. and obtained solid was subjected to drying at about 57° C. to afford the title solid. The resulting dispersion was found to be amorphous by X-ray powder diffraction according to FIG. 7a. The said dispersion is kept at 25° C. under 40% relative humidity for 24 hours and PXRD was recorded and found to be amorphous according to FIG. 7b.

### Example 16

### Preparation of a Solid Dispersion Comprising an Amorphous Form of Eliglustat Hemitartarate

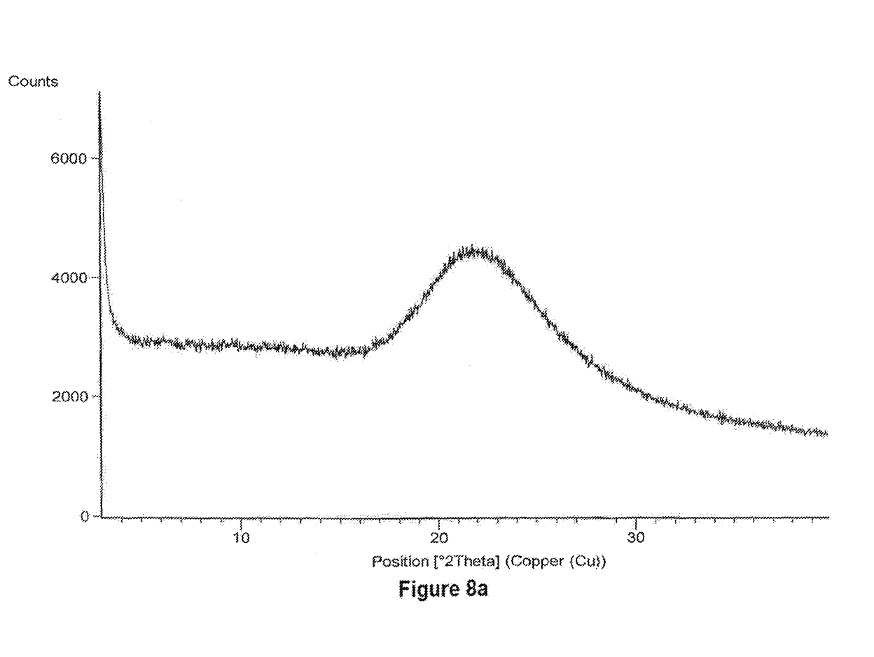

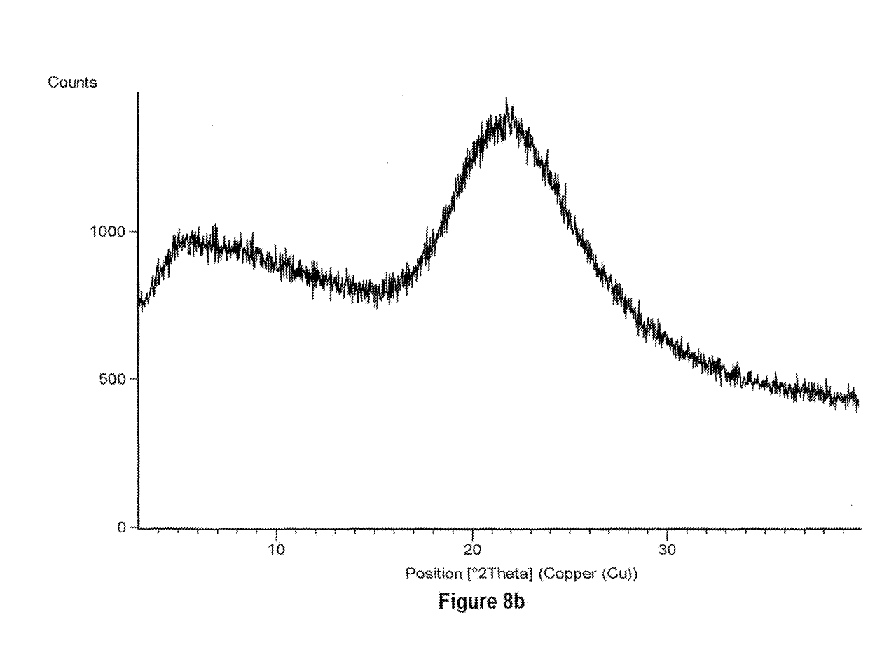

2 g of eliglustat hemitartarate was charged in 40 mL of methanol followed by addition of 2 g of Copovidone. The mixture was stirred for clear solution and filtered to make it particle free, the bed was washed with 20 mL of methanol. Then 2 g of Syloid is added to the filtrate and filtrate is subjected to distillation under reduced pressure at about 57° C. and obtained solid was subjected to drying at about 57° C. to afford the title solid. The resulting dispersion was found to be amorphous by X-ray powder diffraction according to FIG. 8a. The said dispersion is kept at 25° C. under 40% relative humidity for 24 hours and PXRD was recorded and found to be amorphous according to FIG. 8b and D₉₀ of the resultant solid is about 437 microns.

### Example 17

### Preparation of a Solid Dispersion Comprising an Amorphous Form of Eliglustat Hemitartarate and Syloid

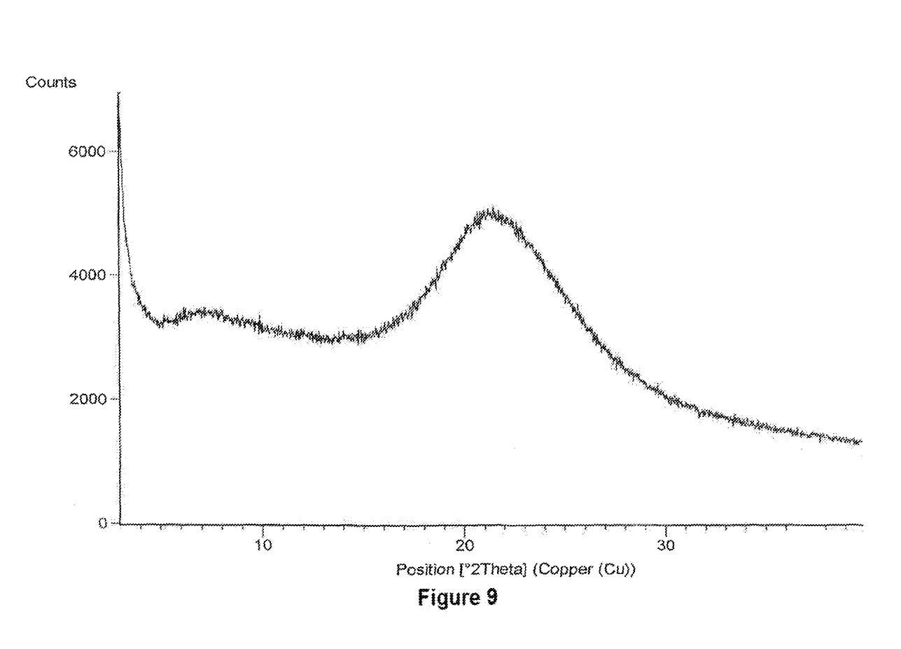

1 g of eliglustat hemitartarate was dissolved in 25 mL of methanol and filtered to make it particle free. Then 1 g of Syloid 244 FPNF was added to the filtrate and solvent from the filtrate was evaporated under reduced pressure at 56° C. and obtained solid was subjected to drying at 56° C. to afford the title solid. The resulting dispersion was found to be amorphous by X-ray powder diffraction according to FIG. 9 and D₉₀ of the resultant solid is about 4 microns.

### Example 18

### Preparation of a Solid Dispersion Comprising an Amorphous Form of Eliglustat Hemitartarate and Syloid

1 g of eliglustat hemitartarate was dissolved in 25 mL of methanol and filtered to make it particle free. Then 500 mg of Syloid 244 FPNF was added to the filtrate and solvent from the filtrate was evaporated under reduced pressure at 56° C. and obtained solid was subjected to drying at 56° C. to afford the title solid. The resulting dispersion was found to be amorphous by X-ray powder diffraction.

1: Eliglustat hemitartrate of Formula I in solid amorphous form.

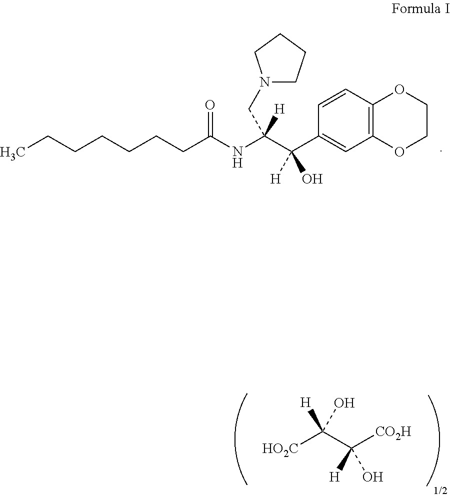

2: A process for preparing amorphous form of Eliglustat hemitartrate comprising:
a) providing a solution of Eliglustat hemitartrate in a solvent;
b) removing the solvent from the solution obtained in step a), and
c) recovering amorphous form of Eliglustat hemitartrate.

3: The process of claim 2 wherein suitable solvent in step a) is selected from alcohols, esters, ketones, hydrocarbons, water or mixtures thereof.

4: The process of claim 2 wherein suitable solvent in step a) is selected from methanol, isopropyl alcohol, ethyl acetate, acetone, dichloromethane or mixtures thereof.

5: The process of claim 2 wherein removal of solvent in step b) is affected by evaporation, freeze drying, spray drying, lyophilization, by addition of suitable anti-solvent or any combination thereof.

6: The process of claim 2 wherein step c) involves an additional step of drying the isolated Eliglustat hemitartrate.

7: A solid dispersion comprising amorphous Eliglustat hemitartrate in a dispersing agent.

8: The solid dispersion of claim 7, wherein the dispersing agent comprises hydroxypropyl methyl cellulose (HPMC), Polyvinyl pyrrolidone (PVP), Colloidal silicon dioxide and the like.

9: The solid dispersion of claim 8, wherein PVP is of different grades like K-15, K-30, K-60 and like.

10: The solid dispersion of claim 8, wherein colloidal silicon dioxide like Syloid 244 FP is employed.

11: A pharmaceutical composition comprising solid dispersion of claim 7.
